(19) United States
(12) Patent Application Publication
(10) Pub. No.: US 2025/0245401 A1
(43) Pub. Date: Jul. 31, 2025

(54) ELECTRONIC DEVICE FOR PERFORMING MOLECULAR DYNAMICS SIMULATION AND METHOD OF OPERATING THE SAME

(71) Applicant: SAMSUNG ELECTRONICS CO., LTD., SUWON-SI (KR)

(72) Inventors: IHOR VASYLTSOV, Suwon-si (KR); ZHEN ZHANG, Suwon-si (KR)

(21) Appl. No.: 18/949,359

(22) Filed: Nov. 15, 2024

(30) Foreign Application Priority Data
Jan. 30, 2024 (KR) 10-2024-0014193

(51) Int. Cl. G06F 30/27

(52) U.S. Cl. CPC G06F 30/27

(57) ABSTRACT

An electronic device for performing a molecular dynamics simulation and a method of operating the same are provided. The electronic device includes at least one processor and a memory configured to store instructions, wherein at least one of the instructions, when executed by the at least one processor, causes the electronic device to split a workload for a molecular simulation into a plurality of partitions corresponding to a plurality of regions of a molecular structure, respectively, map a localized model of a plurality of localized models to each of the plurality of partitions of the workload based on a corresponding region of the plurality of regions of the molecular structure, and perform the molecular simulation based on the mapping of the localized model to each of the plurality of partitions of the workload.

### CROSS-REFERENCE TO RELATED APPLICATION

This application is based on and claims priority under 35 U.S.C. § 119 to Korean Patent Application No. 10-2024-0014193, filed on Jan. 30, 2024, in the Korean Intellectual Property Office, the entire contents of which are incorporated by reference herein in their entirety.

### BACKGROUND

### 1. Field of the Invention

Embodiments relate to an electronic device for performing a molecular dynamics simulation and a method of operating the same.

### 2. Description of the Related Art

A molecular dynamics (MD) simulation may be used in computational research, enabling scientists to model and analyze the behavior of molecules over time. The molecular dynamics simulations provide critical insights into the physical movements and interactions of atoms and molecules, facilitating research and development in a variety of fields, such as chemistry, biology, climate change, civil engineering, mining, pharmacy and drug development, materials science, and semiconductors.

In some cases, MD simulations may be performed using powerful computational resources, often requiring high-performance computing clusters to manage the complex calculations involved. Therefore, as the scale and complexity of the MD simulations increase, there is a need in the art for systems and methods that can provide a more efficient and accessible method to perform MD simulations.

### SUMMARY

The present disclosure describes systems and methods for performing molecular dynamics simulation. Embodiments of the present disclosure may be configured to split a workload for a molecular dynamics simulation into a plurality of partitions. An embodiment may perform the simulation based on mapping a localized model to each partition. Additionally, in some cases, the mapping of the partition model may be dynamically updated based on a metric exceeding a threshold value. The methods and systems of the present disclosure leverage processing capabilities to manage the computational demands of MD simulations.

According to an aspect, there is provided an electronic device including at least one processor and a memory configured to store instructions, wherein at least one of the instructions, when executed by the at least one processor, may cause the electronic device to split a workload for a molecular simulation into a plurality of partitions corresponding to a plurality of regions of a molecular structure, respectively, map a localized model of a plurality of localized models to each of the plurality of partitions of the workload based on a corresponding region of the plurality of regions of the molecular structure, and perform the molecular simulation based on the mapping of the localized model to each of the plurality of partitions of the workload.

The workload is split into the plurality of partitions based on a computational complexity of the plurality of localized models.

The instructions, when executed by the at least one processor, may cause the electronic device to, determine that a computational complexity of the localized model is greater than a computation threshold, and split a corresponding partition of the plurality of partitions into a plurality of sub-partitions based on the determination.

The instructions, when executed by the at least one processor, may cause the electronic device to compute a metric for a corresponding partition of the plurality of partitions, determine whether a change in the metric is greater than a metric threshold, and iteratively update the localized model based on the determination.

The instructions, when executed by the at least one processor, may cause the electronic device to perform the molecular simulation for the corresponding partition using a tabular model based on an adjacent partition when the change in the metric is less than the metric threshold.

The metric may include at least one of an accuracy of the localized model, an atom in the corresponding partition, a ghost atom in the corresponding partition, a radial distribution function (RDF), an angular distribution function (ADF), temperature and pressure of the corresponding partition, and a motion of atoms in the corresponding partition.

The plurality of localized models may include localized models of different computational complexities.

The at least one processor includes a plurality of homogeneous processors, and a higher computational complexity of a corresponding localized model results in a smaller size of a corresponding partition.

The instructions, when executed by the at least one processor, may cause the electronic device to determine a processor among a plurality of heterogeneous processors, and allocate the processor to the localized model based on a computational complexity of the localized model.

The instructions, when executed by the at least one processor, may cause the electronic device to determine an absence of an atomic interaction in the corresponding partition and switch off an interaction parameter for the corresponding partition.

According to another aspect, there is provided a method of operating an electronic device, the method including splitting a workload for a molecular simulation into a plurality of partitions corresponding to a plurality of regions of a molecular structure, respectively, mapping a localized model of a plurality of localized models to each of the plurality of partitions of the workload based on a corresponding region of the plurality of regions of the molecular structure, and performing the molecular simulation based on the mapping of the localized model to each of the plurality of partitions of the workload.

According to another aspect, there is provided a method of performing a molecular simulation, the method including computing a metric for each of a plurality of partitions of a workload for a molecular simulation, selecting a localized model for each of the plurality of partitions based on the metric, and performing a molecular simulation using the selected localized model for each of the plurality of partitions.

Additional aspects of embodiments will be set forth in part in the description which follows and, in part, will be apparent from the description, or may be learned by practice of the disclosure.

### BRIEF DESCRIPTION OF THE DRAWINGS

These and/or other aspects, features, and advantages of the invention will become apparent and more readily appreciated from the following description of embodiments, taken in conjunction with the accompanying drawings:

FIGS. 1 and 2 illustrate a workload, a partition, and a localized model for a molecular simulation, according to an embodiment;

FIG. 3 illustrates an operation of performing a molecular simulation, according to an embodiment;

FIGS. 4 and 5 illustrate a partition-model mapping operation in a homogeneous hardware environment, according to an embodiment;

FIGS. 6 and 7 illustrate a partition-model mapping operation in a heterogeneous hardware environment, according to an embodiment;

FIGS. 8 and 9 illustrate a table format-based model according to an embodiment;

FIG. 10 illustrates an electronic device according to an embodiment; and

FIG. 11 illustrates a method of operating an electronic device, according to an embodiment.

FIG. 12 illustrates a method of performing a molecular simulation, according to an embodiment.

### DETAILED DESCRIPTION

The present disclosure provides a method for performing molecular dynamics simulations. Embodiments of the present disclosure provide a system for executing MD simulations, while incorporating advanced hardware and software optimizations to enhance performance. The methods and systems of the present disclosure leverage processing capabilities to manage the computational demands of MD simulations.

Despite the advancements in computational power, performing MD simulations remains resource-intensive and time-consuming. In some cases, presently used systems often suffer from limitations in scalability, efficiency, and accessibility. Additionally, high-performance computing clusters may be expensive to maintain and operate, and thus the use of such clusters may be confined to large research institutions. Moreover, the complexity of setting up and managing such simulations presents a significant barrier to entry for many researchers and small organizations.

The present disclosure describes systems and methods for performing molecular dynamics simulation. Embodiments of the present disclosure may be configured to split a workload for a molecular dynamics simulation into a plurality of partitions. An embodiment may perform the simulation process based on mapping a localized model to each partition. Additionally, in some cases, the mapping of the partition model may be dynamically updated based on a metric exceeding a threshold value.

In some cases, the molecular simulation for each of the plurality of partitions in the workload may be performed using a localized model. In some cases, the localized model of the plurality of localized models may be mapped to the partitions based on the computational complexity of the model. According to an embodiment, the localized model may be a machine learning force field (MLFF) model. In some cases, the partitioning of the workload may be performed and dynamically updated based on the localized models.

In some cases, a partition-model mapping may be performed that maps each partition model to a corresponding partition based on the computational complexity of the models. In some cases, the mapping may be performed based on a location of the atoms within each partition and a metric may be computed for the corresponding partition. According to an embodiment, a change in the metric may be computed to provide insights into the presence of a ghost atom or an atom that may have moved to an adjacent partition. In some cases, the partition-model mapping may be dynamically updated based on the computed metric.

In some cases, embodiments of the present disclosure may be implemented in a form of a software to provide for distributed and/or parallel (e.g., faster) simulation of a molecular dynamics workload. An exemplary embodiment of the present disclosure may provide for a large-scale atomic/molecular massively parallel simulator (LAMMPS) for dynamically changing chemical structures for a semiconductor application. For example, the changing chemical structures may include dry or wet etching techniques. In some cases, an exemplary embodiment may be applied to a supercomputer system.

The present disclosure describes systems and methods for performing a molecular dynamics simulation. Embodiments of the present disclosure may be configured to split a workload for a molecular simulation into a plurality of partitions corresponding to a plurality of regions of a molecular structure, respectively. In some cases, a localized model, such as a MLFF model, may be mapped to each of the plurality of partitions of the workload based on a corresponding region of the plurality of regions of the molecular structure. According to an embodiment, the molecular simulation may be performed based on the mapping of the localized model to each of the plurality of partitions of the workload.

Further, an embodiment of the present disclosure may provide a method for performing a molecular simulation operation. In some cases, the method comprises computing a metric for each of a plurality of partitions of a workload for a molecular simulation. Next, the method includes selecting a localized model for each of the plurality of partitions based on the metric. The molecular simulation is performed using the selected localized model for each of the plurality of partitions.

Accordingly, by incorporating hardware and software optimizations, embodiments of the present disclosure are able to provide fast and more accurate results. An embodiment of the disclosure uses a partition model that enables acceleration of the simulation operation while maintaining the accuracy of the simulation. In some cases, the partition model mapping may be dynamically updated by allocating a localized model appropriate for the changed metric. In some cases, embodiments may be used in platforms with limited hardware resources to perform process and/or thread-based parallel simulations of molecular dynamics tasks (such as a small computer center, local personal computer, etc.). Additionally, embodiments may ensure an ease of use and broad accessibility for researchers across various fields. In some cases, the methods and systems described herein enable a reduction in the computational cost and complexity of performing MD simulations while providing a computational tool for widespread adoption and accelerated scientific discovery.

The following detailed structural or functional description is provided as an example only and various alterations and modifications may be made to the embodiments. Accordingly, the embodiments are not construed as limited to the disclosure and should be understood to include all changes, equivalents, and replacements within the idea and the technical scope of the disclosure.

As used herein, “A or B”, “at least one of A and B”, “at least one of A or B”, “A, B or C”, “at least one of A, B and C”, “at least one of A, B, or C”, and “one or a combination of at least two of A, B, and C,” each of which may include any one of the items listed together in the corresponding one of the phrases, or all possible combinations thereof. Although terms, such as first, second, and the like are used to describe various components, the components are not limited to the terms. These terms should be used only to distinguish one component from another component. For example, a first component may be referred to as a second component, and similarly the second component may also be referred to as the first component.

It should be noted that if one component is described as being “connected”, “coupled”, or “joined” to another component, a third component may be “connected”, “coupled”, and “joined” between the first and second components, although the first component may be directly connected, coupled, or joined to the second component.

The singular forms “a”, “an”, and “the” are intended to include the plural forms as well, unless the context clearly indicates otherwise. It will be further understood that the terms “comprises/comprising” and/or “includes/including” when used herein, specify the presence of stated features, integers, steps, operations, elements, and/or components, but do not preclude the presence or addition of one or more other features, integers, steps, operations, elements, components and/or groups thereof.

Unless otherwise defined, all terms, including technical and scientific terms, used herein have the same meaning as commonly understood by one of ordinary skill in the art to which the present disclosure pertains. Terms, such as those defined in commonly used dictionaries, should be construed to have meanings matching with contextual meanings in the relevant art, and are not to be construed to have an ideal or excessively formal meaning unless otherwise defined herein.

Hereinafter, embodiments will be described in detail with reference to the accompanying drawings. When describing the embodiments with reference to the accompanying drawings, like reference numerals refer to like components and a repeated description related thereto will be omitted.

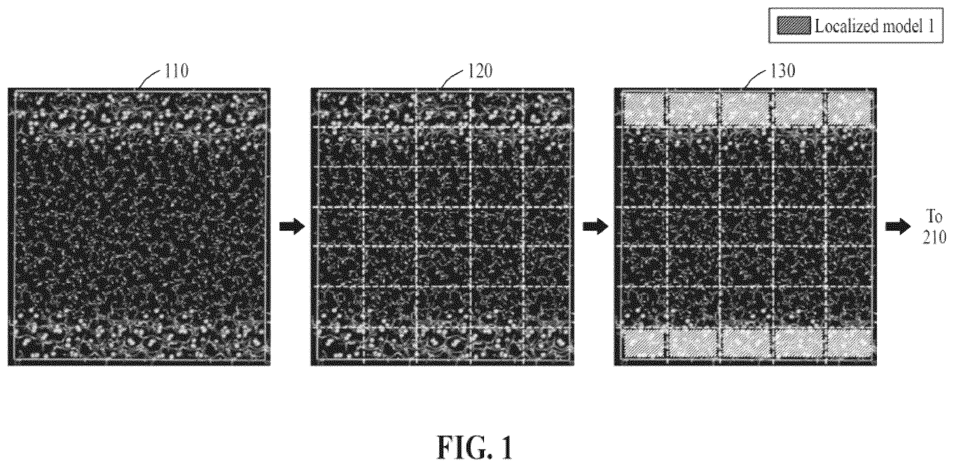

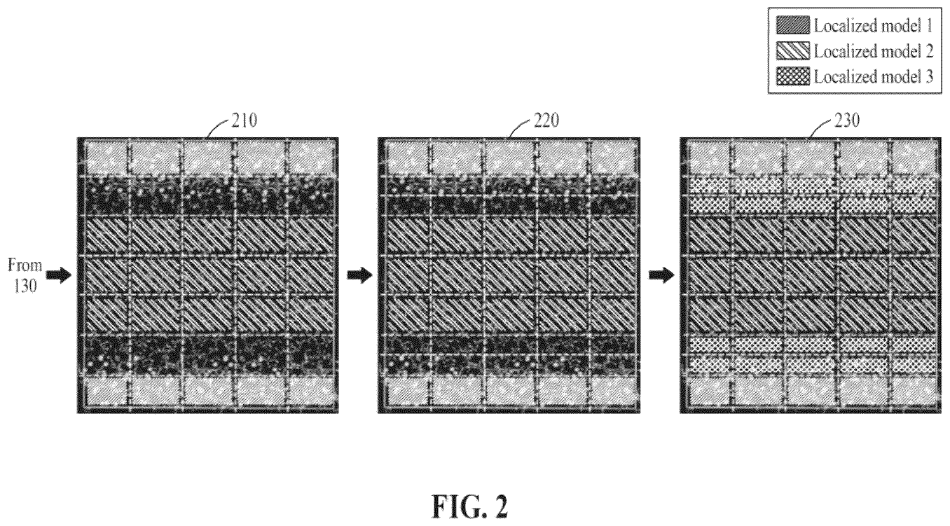

FIGS. 1 and 2 illustrate a workload, a partition, and a localized model for a molecular simulation, according to an embodiment.

A molecular dynamics simulation is used to calculate the dynamics of atoms by numerically solving Newton's equation of motion, given a potential or force between atoms in the physical world. For example, the molecular dynamics simulation may be an important tool for research and development in various fields, such as chemistry, biology, climate change, civil engineering, mining, pharmacy and drug development, materials science, and semiconductors. For example, a molecular dynamics simulation may include three operations of 1) forward/reverse communication, 2) computation, and 3) modification. Among operations of the molecular dynamics simulation, operations using processors (e.g., hardware cores or electronic devices) may be occupied by computation operations, and data communication between processors (e.g., hardware cores or electronic devices) may occur in the forward/reverse communication operation. In case of the forward/reverse communication operation, coordinate data and force data of a molecule may be moved between processors (e.g., hardware cores or electronic devices).

The molecular dynamics simulation may be performed based on a spatial-decomposition algorithm with atoms distributed into a plurality of domains. For example, the molecular dynamics simulation may include a significant amount of computations, as a result, performing the simulation with a single core or a single processor may be difficult. Accordingly, the simulation may be performed based on a plurality of cores, a plurality of processors, or a plurality of electronic devices. As used herein, a domain may refer to a unit that may be divided when a work or a job is performed based on distribution over multiple cores, processors, or electronic devices. Hereinafter, for ease of description, a molecular dynamics simulation may be referred to as a molecular simulation. Additionally, the sum of domains in which the molecular dynamics simulation is performed (that is, the complete domain) may be referred to as a workload. Additionally, an individual domain may be referred to as a partition or a space.

A molecular simulation may be performed by a single general model or a plurality of localized models. As used herein, a general model may refer to a model that has a larger size and a slower computation speed than a localized model and may be a general-purpose model that may not be limited to a specific task. Additionally, the localized model may refer to a lightweight model that may have a smaller size and a faster computation speed than the general model. In some cases, the localized model may operate in a small-scale workload and may indicate a model that may process a limited simulation area.

According to an embodiment of the present disclosure, a method of performing a molecular simulation using a localized model is described. In some examples, a plurality of localized models may be used rather than using a single general model. The molecular simulation may be performed efficiently by splitting a workload for the molecular simulation into a plurality of partitions and then mapping a localized model to each partition.

FIG. 1 shows a workload for a molecular simulation 110. The workload 110 may include areas with various properties (e.g., various atoms and interatomic interactions).

As an example shown in FIG. 1, silicon-related atoms may be arranged (e.g., are mainly arranged) at the top and the bottom of the workload and water-related atoms are arranged in the middle of the workload.

In some cases, the workload may be split into a plurality of partitions as shown in 120. The plurality of partitions may be split into the same or different sizes and/or shapes. For ease of description, FIG. 1 illustrates an example in which a plurality of partitions may be split into the same size and shape. However, embodiments are not limited thereto.

In some cases, a first localized model 130 may be matched to each of the top and the bottom partitions in which silicon-related atoms may be arranged. For example, the first localized model may be a model appropriate for a molecular simulation of silicon-related atoms that are mainly arranged at the top and bottom of the workload.

Further, referring to FIG. 2, a second localized model 210 may be matched to each of the middle partitions in which water-related atoms may be arranged, e.g., water-related atoms are mainly arranged in the middle partition of the workload in FIGS. 1-2. For example, the second localized model 210 may be a model appropriate for a molecular simulation of water-related atoms.

An intermediate model 220 depicts remaining partitions in which the first localized model and the second localized model are not matched. In some cases, the intermediate model 220 may refer to an area where silicon-related atoms and water-related atoms are mixed or combined. In some cases, the corresponding partition may be split into two sub-partitions since computational complexity of a molecular simulation for the partition in which silicon-related atoms and water-related atoms are mixed is higher than that of the other partitions.

In some cases, a third localized model 230 may be matched to each of the split sub-partitions. For example, the third localized model 230 may refer to a model with higher computational complexity than other localized models.

In some cases, by periodically performing operation of the steps shown in FIGS. 1-2, the localized model mapped to each partition may be updated.

In some cases, a molecular simulation may be efficiently performed using various computational precision through space-domain parallelism. Space-domain parallelism refers to a parallel computing technique where the simulation space (the physical region containing the atoms and molecules) may be divided into smaller subdomains or regions. Each subdomain may be then assigned to a different processor or a computing node, which independently computes the interactions and movements of the particles within an assigned region. The parallel computing technique provides for efficient scaling of simulations across multiple processors, enabling the study of larger systems and longer time scales than would be feasible on a single processor.

As described herein, the space-domain parallelism may refer to a method in which an appropriate localized model may be mapped to each of the plurality of partitions. Additionally, a time-domain parallelism may refer to a method in which the localized model that may be mapped to each partition is periodically updated. In some cases, the complete simulation process may be further accelerated by applying various localized models to different partitions, i.e., considering the computational complexity of a specific partition simulation and the number of atomic particles to be simulated. According to an exemplary embodiment, the localized model may be a machine learning force field (MLFF) model. However, embodiments may not be limited thereto.

A machine learning force field (MLFF) model uses machine learning techniques to predict the potential energy and forces acting on atoms in molecular systems. Trained on data from quantum mechanical calculations, such as Density Functional Theory (DFT), MLFF models capture the complex relationships between atomic configurations and the corresponding energies and forces. The models employ algorithms like neural networks and Gaussian process regression, offering efficient and accurate predictions compared to traditional quantum methods. MLFF models enable large-scale molecular dynamics (MD) simulations, making the models suitable for applications in materials science, drug discovery, etc. The MLFF models provide high-precision results at a fraction of the computational cost, facilitating the study of large systems and time scales.

According to an embodiment of the present disclosure, the molecular simulation operation may be implemented as software that may perform a distributed/parallel simulation (e.g., the simulation may be performed more quickly) on a molecular dynamics workload (e.g., a large-scale atomic/molecular massively parallel simulator (LAMMPS) platform) of chemical structures (e.g., dry/wet etching) that may change dynamically for various semiconductor application fields.

LAMMPS is a software for molecular dynamics (MD) simulations. LAMMPS is designed to run efficiently on parallel computing architectures, making it suitable for simulating large-scale atomic, molecular, and mesoscale systems. The software LAMMPS supports parallel execution using MPI (Message Passing Interface), providing for simulations to scale across thousands of processors. In some cases, LAMMPS provides multiple interatomic potentials (force fields) to model interactions between particles. LAMMPS can perform molecular dynamics, energy minimization, Monte Carlo, and other types of simulations, covering both classical and quantum mechanical models.

Additionally, the molecular simulation operation may be performed by a supercomputer system equipped with a central processing unit (CPU), a graphics processing unit (GPU), and other accelerators in a distributed simulation environment. In some cases, power consumption of the supercomputer system may be reduced. Furthermore, the molecular simulation operation may be performed on other platforms (e.g., a small computer center and a local personal computer (PC)) with limited hardware resources to implement a process/thread-based parallel simulation of molecular dynamics tasks.

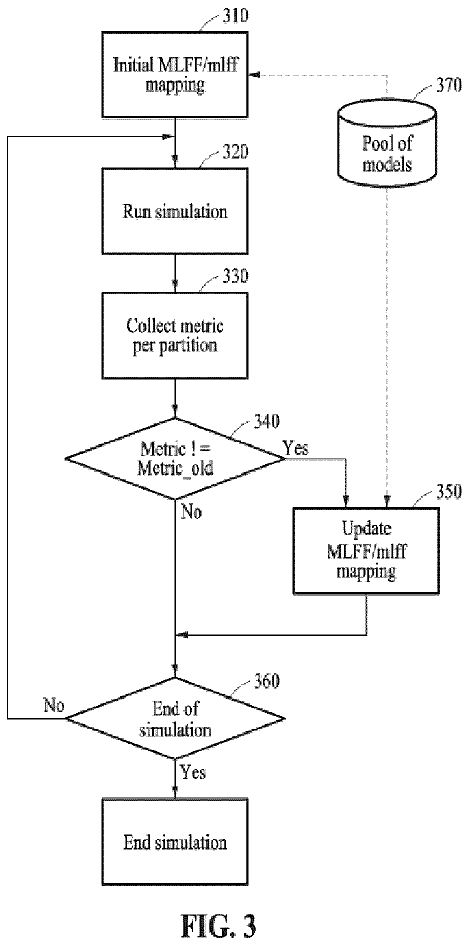

FIG. 3 illustrates an operation of performing a molecular simulation, according to an embodiment.

FIG. 3 shows a flowchart illustrating a molecular simulation operation based on hybrid time and space domain parallelization. MLFF and mlff shown in FIG. 3 represent different localized models. In some cases, MLFF (in upper case) may represent a localized model with relatively high computational complexity. In some cases, mlff (in lower case) may represent a localized model with relatively low computational complexity. However, this is for ease of description. A gradation of the localized model may be arbitrary (that is, the level of computational complexity may be diverse), and the localized models utilized may not be limited to the described example. In some examples, simulation precision may be determined in a single operation or in multiple operations.

Referring to FIG. 3, in operation 310, an initial partition-model mapping may be performed. In molecular dynamics (MD) simulations, initial partition model mapping includes dividing the simulation space into small regions and assigning the small regions to different processors. The process ensures an even distribution of the computational load, optimizing performance by minimizing inter-processor communication and maximizing parallel execution. Each processor handles the interactions and movements of particles within the assigned region, while reducing the need for data exchange between processors. The method enhances scalability, providing for large systems and long simulation times. Efficient initial partition mapping is crucial for achieving balanced workloads and increased computational efficiency in large-scale MD simulations.

According to an embodiment, the initial partition-model mapping may be performed based on a location (e.g., a three-dimensional location) of atoms located within each partition and a metric related to the corresponding partition. A pool of a plurality of localized models 370 may include models that may be used for a molecular simulation. In some cases, a corresponding pool may include various localized models.

The metric corresponding to the partition may include at least one of accuracy of a corresponding localized model, a type of an atom in a corresponding partition, a ghost atom, a radial distribution function (RDF), an angular distribution function (ADF), temperature and pressure of the corresponding partition, and speed, force, and energy of atoms in the corresponding partition.

In molecular simulations, a ghost atom refers to a replicated atom from a neighboring subdomain used to ensure accurate calculations at the boundaries of the simulation partitions. The ghost atoms enable maintaining continuity and accurate force calculations for atoms near the edges of the divided regions. Ghost atoms provide for each processor to compute interactions that span subdomain boundaries without constant communication with other processors, thus enhancing computational efficiency and accuracy in parallel MD simulations.

RDF provides information of the variation of particle density as a function of distance from a reference particle in a system. The RDF provides insights into the structure and arrangement of particles by showing the probability of finding a particle at a distance r from another particle, relative to a completely random distribution. RDF may be used for studying the local structure in liquids, gases, and amorphous solids, revealing information about short-range order and spatial correlations within the system.

ADF characterizes the distribution of angles between triplets of particles in a system, providing information on the angular correlations and geometric arrangements. ADF provides an understanding of the preferred angles formed by neighboring atoms or molecules. In some cases, ADF may be used for analyzing the local structure and bonding patterns in molecular systems. ADF provides an understanding of materials with directional bonding, such as water molecules, polymers, and complex crystal structures, while generating insights into the spatial orientation of particles.

In operation 320, a molecular simulation may be performed based on the partition-model mapping.

In operation 330, a metric of each partition may be collected.

In operation 340, the method may include determination of whether the metric of each partition has changed by more than a predetermined degree. In some cases, the predetermined degree includes a specific ratio or a numerical value. In some cases, the predetermined degree may be predetermined by a user or determined according to the degree of evolution of the molecular simulation. However, embodiments may not be limited thereto. For example, a change in the metric by more than the predetermined degree may indicate that a ghost atom located at the boundary of an adjacent partition may be new. In some examples, the metric change may indicate movement of a local atom (or a plurality of local atoms) located within a corresponding partition to an adjacent partition. However, embodiments are not limited to the described examples.

Operation 350 may be performed (e.g., subsequently) for a partition in which the metric changes by more than the predetermined degree. In some cases, operation 360 may be performed (e.g., subsequently) for a partition in which the metric does not change by more than the predetermined degree.

Referring to FIG. 3, in operation 350, the partition-model mapping may be updated for the partition in which the metric changes by more than the predetermined degree. For example, a localized model that is heavier (such as a localized model that may be larger) than a previous localized model may be mapped to a partition that includes a new ghost atom. Additionally, for example, a localized model that is lighter (such as a localized model that may be smaller) than a previous localized model may be mapped to a partition in which a localized atom (e.g., a plurality of localized atoms) may have moved to an adjacent partition.

In operation 360, the system may determine whether the molecular simulation has ended. When the molecular simulation has not ended, operation 320 may be performed.

Accordingly, the partition-model mapping may be dynamically updated by mapping a localized model appropriate for the changed metric even though the metric of some partitions changes as the simulation is performed. A simulation operation may be accelerated as a high simulation accuracy may be maintained and a large-scale workload including semiconductor evolving structures (e.g. etching) may be effectively simulated.

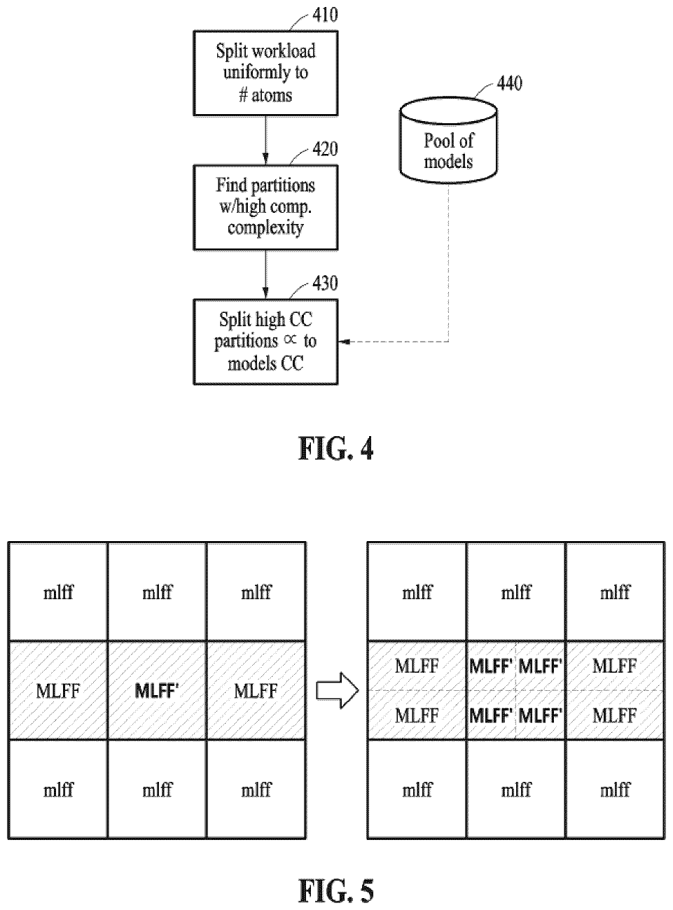

FIGS. 4 and 5 illustrate a partition-model mapping operation in a homogeneous hardware environment, according to an embodiment.

FIG. 4 illustrates a partition-model mapping operation in a homogeneous hardware environment (e.g., the same CPUs or the same accelerators). When a molecular simulation is performed by a plurality of homogeneous processors, a partition corresponding to a localized model with high computational complexity may be split into a size smaller than a partition mapped to a localized model with low computational complexity.

Referring to FIG. 4, in operation 410, a workload may be split uniformly into a plurality of partitions according to the number of atoms. In some cases, an initial workload split may be performed uniformly according to the number of atoms. In some cases, the initial workload split may be based on a location (e.g., a three-dimensional location) of atoms located within each partition and a metric related to the corresponding partition.

In operation 420, the system may search for a partition from among the plurality of partitions in which computational precision is greater than or equal to a predetermined threshold.

In operation 430, the partition in which the computational precision is greater than or equal to the predetermined threshold may be split into sub-partitions according to computational complexity of a model allocated to a corresponding partition. For example, in case of a high computational complexity of the allocated model, the corresponding partition may be split into small-sized sub-partitions. A pool of a plurality of localized models 440 may include models that may be used for a molecular simulation. In some cases, a corresponding pool may include various localized models.

Accordingly, the workload may be balanced in proportion to the computational complexity of the model, thereby further improving the speed of a split simulation.

Referring to FIG. 5, MLFF may be assumed to have twice the computational complexity of mlff. Additionally, MLFF′ may have four times the computational complexity of mlff. Since MLFF has twice the computational complexity of mlff, each MLFF may be allocated to sub-partitions that may be half of the partitions to which an mlff is allocated (i.e., sub-partitions of the MLFF are one half times smaller than a partition to which mlff is allocated). Similarly, since MLFF′ has four times the computational complexity of mlff, each MLFF′ may be allocated to sub-partitions that may be a one fourth of the partitions to which an mlff is allocated (i.e., sub-partitions of the MLFF′ that are ¼ times smaller than a partition to which mlff is allocated). As shown in FIG. 5, mlff, MLFF, and MLFF′ may represent localized models with various computational complexities, for ease of description. Additionally, other localized models with different computational complexities may be used without limitation.

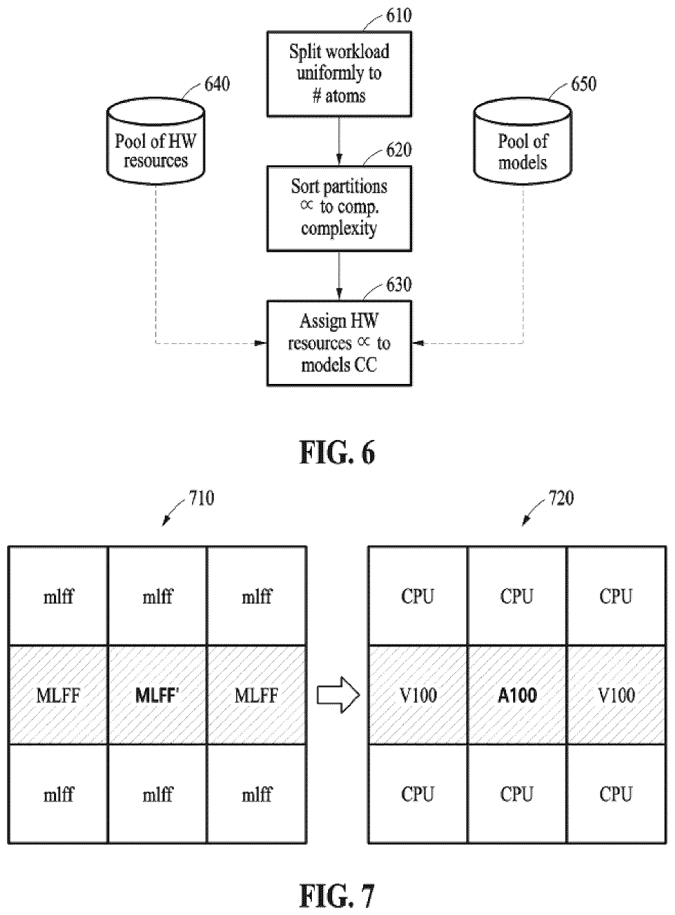

FIGS. 6 and 7 illustrate a partition-model mapping operation in a heterogeneous hardware environment, according to an embodiment.

FIG. 6 shows a flowchart illustrating a partition-model mapping operation in a heterogeneous hardware environment (e.g., a CPU and a GPU). In some cases, a partition-model mapping may be performed according to computational characteristics of a hardware resource when a molecular simulation is performed by a plurality of heterogeneous processors.

In operation 610, a workload may be split uniformly into a plurality of partitions according to the number of atoms. In some cases, an initial workload split may be performed uniformly according to the number of atoms. In some cases, the initial workload split may be based on a location (e.g., a three-dimensional location) of atoms located within each partition and a metric related to the corresponding partition.

In operation 620, the plurality of partitions may be sorted according to computational complexity of a model allocated to each partition.

In operation 630, a hardware resource may be allocated to a corresponding partition according to the computational complexity of the model allocated to each partition. For example, a high-performance hardware resource may be allocated to a partition allocated to a model with high computational complexity. Additionally, for example, a low-performance hardware resource may be allocated to a partition that is allocated to a model with low computational complexity. A pool of a plurality of hardware resources 640 may include available heterogeneous hardware resources. A pool of a plurality of localized models 650 may include models that may be used for a molecular simulation. In some cases, a corresponding pool may include various localized models.

Accordingly, the workload may be balanced in proportion to the computational characteristics of hardware resources and the computational complexity of the model, thereby further improving the speed of a split simulation.

Referring to FIG. 7, MLFF may be assumed to have twice the computational complexity of mlff and MLFF′ may be assumed to have four times the computational complexity of mlff. Workload 710 may represent models allocated to each partition and workload 720 may represent hardware resources allocated to each partition.

In some cases, V100 including higher performance than a CPU allocated to a partition of mlff may be allocated to a partition of MLFF since MLFF has twice the computational complexity of mlff. Additionally, A100 including higher performance than the V100 allocated to a partition of MLFF may be allocated to a partition of MLFF′ since MLFF′ has twice the computational complexity of MLFF. As shown in FIG. 5, mlff, MLFF, MLFF′, may represent localized models with various computational complexities and CPU, V100, and A100 may represent hardware resources with various performances, for ease of description. However, embodiments may not be limited thereto and a plurality of other localized models with different computational complexities and hardware resources of different performances may be used without limitation.

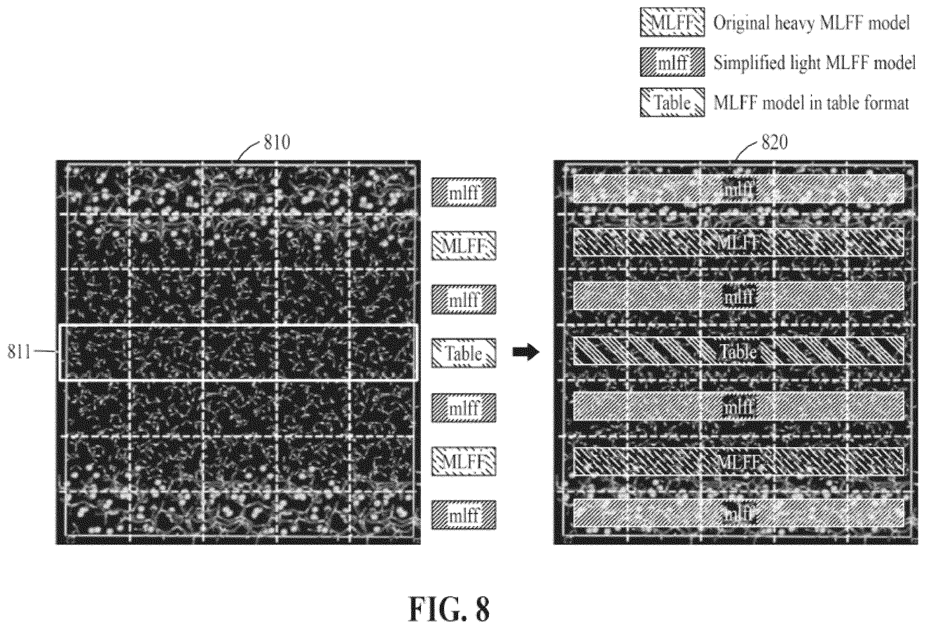

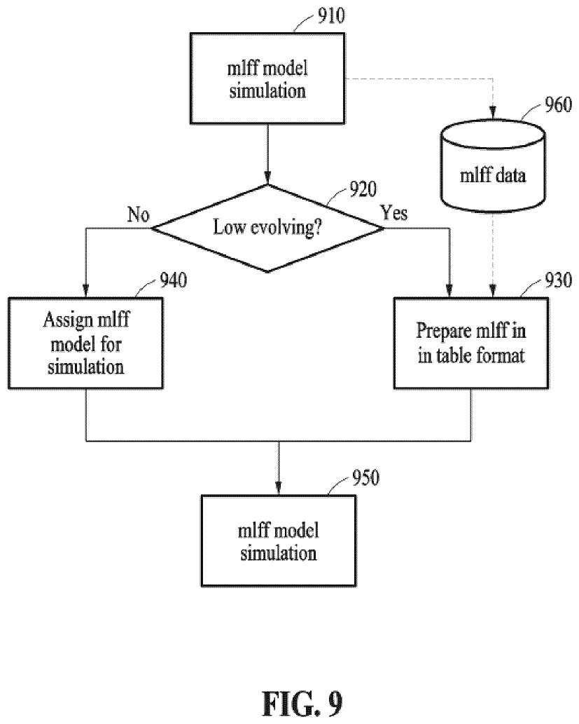

FIGS. 8 and 9 illustrate a table format-based model according to an embodiment.

FIG. 8 shows a workload for a molecular simulation 810. For example, in case of a large-scale workload, areas 811 may include a low evolving ratio (i.e., areas that have no significant change in structure for a sufficient period of time). The molecular simulation may be performed using a tabular model determined based on a simulation result of an adjacent partition for area(s) 811 (i.e., instead of directly performing an operation related to a molecular simulation). Additionally, FIG. 8 shows localized models 820 allocated to each partition. In some cases, a tabular model may be allocated to areas 811.

In some cases, the total simulation speed may be increased by reducing repeated calculations since the tabular model may be determined based on the simulation result of an adjacent partition (e.g., instead of calculating directly). For example, the tabular model may be determined based on a method of averaging the simulation result of an adjacent partition. However, embodiments may not be limited thereto.

Referring to FIG. 9, in operation 910, simulation result data 960 may be accumulated as a general molecular simulation.

In operation 920, the system may determine whether a partition with a low evolving ratio exists within a workload. For example, a partition with a low evolving ratio exists when a simulation result does not change by more than a predetermined degree over time. However, embodiments are not limited to the described example. For example, operation 930 may be performed subsequently for the corresponding partition if the partition with a low evolving ratio exists. In some examples, operation 940 may be performed subsequently for the remaining partitions.

In operation 930, a tabular model that may be used for the partition with a low evolving ratio may be generated based on the accumulated simulation result data 960. Additionally, a molecular simulation may be performed on the partition based on the tabular model.

In operation 940, a molecular simulation for performing direct computation may be performed on the remaining partitions in which evolving ratios are not low.

In operation 950, a next operation of the molecular simulation may be performed sequentially.

According to another embodiment, the same approach may be applied to calculation of the models including traditional potentials. For example, when an atomic interaction does not exist in a particular partition (or when an atomic interaction simulation is not required), pair potential such as pair_coeff I J none (Vᵢⱼ(none)) may be used to omit computation of some atomic interactions. As used herein, pair potential, pair_coeff I J none (Vᵢⱼ(none)) may be a description of pair_style hybrid command to avoid calculation on some atom pairs and may include, for example, a pair_style hybrid command of LAMMPS.

In case of LAMMPS, a pair potential may refer to a mathematical function describing the interaction energy between pairs of atoms based on their separation distance. For example, pair potential may include Lennard-Jones (LJ) Potential that models van der Waals interactions, Coulomb potential that describe electrostatic interactions between charged particles, and Morse potential that may be used for bonded interactions, particularly in diatomic molecules. In some cases, pair potential may be defined based on parameters such as interaction strength and equilibrium distances for determining atomic forces and movements in simulations.

Additionally, the pair_style hybrid command in LAMMPS facilitates the integration of multiple pair potential styles within a single molecular simulation which may be used to accurately model systems with diverse types of interactions, providing for different potential models to be applied to specific atom pairs. The command enhances simulation flexibility and precision by combining distinct interaction models suited to various types of atomic interactions. To utilize the Lennard-Jones potential for van der Waals interactions and the Embedded Atom Method (EAM) for metallic bonding within the same simulation, users may specify a combination of the potentials. For instance, the Lennard-Jones potential may be assigned to interactions between a specific type of atom and the EAM potential may be assigned to each type of atoms. The pair_style hybrid command may be used for simulating complex systems such as alloys, biomolecular assemblies, and composite materials.

Accordingly, a heavy model may be used in a light mode. In some cases, a complex, resource-intensive model (heavy model) is being utilized in a simplified, less demanding manner (light mode), such as by reducing the number of variables, interactions, or processes considered. For example, computation of the heavy model may be simplified by switching off an atom-to-atom interaction (or e.g., a plurality of atom-to-atom interactions). In some cases, a ghost atom analysis or sub-space mapping may be performed. In some cases, the method of FIG. 9 may be applied to an existing model, thereby simplifying solution migration and speeding up the simulation.

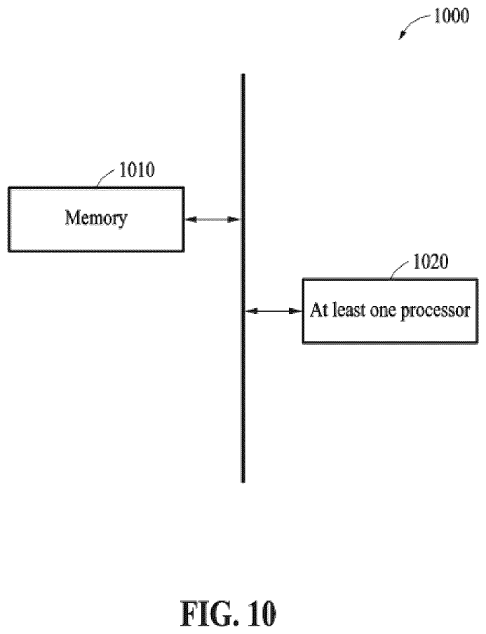

FIG. 10 illustrates an electronic device according to an embodiment.

Referring to FIG. 10, an electronic device 1000 according to an embodiment may include a memory 1010 and at least one processor 1020. The memory 1010 and the at least one processor 1020 may communicate with each other through a bus, peripheral component interconnect express (PCIe), and/or a network on a chip (NoC).

The memory 1010 may include computer-readable instructions. The memory 1010 may cause the electronic device 1000 to perform the aforementioned operations, as at least one of the instructions stored in the memory 1010 is executed by the at least one processor 1020. The memory 1010 may be a volatile memory or a non-volatile memory.

Memory 1010 includes one or more memory devices. Examples of a memory device include random access memory (RAM), read-only memory (ROM), or a hard disk. Examples of memory devices include solid state memory and a hard disk drive. In some examples, memory is used to store computer-readable, computer-executable software including instructions that, when executed, cause at least one processor 1020 to perform various functions described herein.

In some cases, memory 1010 includes a basic input/output system (BIOS) that controls basic hardware or software operations, such as an interaction with peripheral components or devices. In some cases, memory 1010 includes a memory controller that operates memory cells of memory 1010. For example, the memory controller may include a row decoder, column decoder, or both. In some cases, memory cells within memory 1010 store information in the form of a logical state.

The at least one processor 1020 may be a device that executes instructions or programs or controls the electronic device 1000 and may include, for example, a CPU and/or a GPU. In some cases, at least one processor 1020 is configured to operate a memory array using a memory controller. In other cases, a memory controller is integrated into the at least one processor 1020. In some cases, processor 1020 is configured to execute computer-readable instructions stored in memory 1010 to perform various functions. In some aspects, processor 1020 includes special purpose components for modem processing, baseband processing, digital signal processing, or transmission processing. According to some aspects, processor 1020 comprises the one or more processors described herein.

As described, the electronic device 1000 may split a workload for a molecular simulation into a plurality of partitions spatially, may map one of a plurality of localized models to each of the plurality of partitions, and may perform the molecular simulation using a localized model applied to each of the plurality of partitions.

The electronic device 1000 may split the workload into the plurality of partitions according to a computational complexity of the plurality of localized models. Additionally, when the computational complexity of a localized model to be mapped is greater than a predetermined threshold, the electronic device 1000 may split a corresponding partition into two or more sub-partitions.

Furthermore, the electronic device 1000 may update a localized model mapped to a reference partition in which a metric changes by more than a predetermined degree among the plurality of partitions. The metric may include at least one of accuracy of a corresponding localized model, a type of an atom in a corresponding partition, a ghost atom, an RDF, an ADF, temperature and pressure of the corresponding partition, and speed, force, and energy of atoms in the corresponding partition.

The plurality of localized models may include localized models with different computational complexities.

When the at least one processor includes a plurality of homogeneous processors, in case of a high computational complexity of a corresponding localized model, a corresponding partition may be split. Additionally or alternatively, when the at least one processor includes a plurality of heterogeneous processors, the electronic device 1000 may allocate any one of the plurality of heterogeneous processors to execute a corresponding localized model according to computational complexity of the corresponding localized model. Additionally or alternatively, a molecular simulation for a reference partition, among the plurality of partitions, in which a metric changes over time is less than or equal to a predetermined threshold may be performed by a tabular model determined based on a simulation result of partitions adjacent to the reference partition. Additionally or alternatively, an interaction between atoms for a reference partition, among the plurality of partitions, in which an atomic interaction does not exist may be switched off.

The electronic device 1000 may include, for example, various computing devices, such as a mobile phone, a smartphone, a tablet PC, an e-book device, a laptop, a PC, a desktop, a workstation, or a server, various wearable devices, such as a smart watch, smart eyeglasses, a head-mounted display (HMD), or smart clothing, various home appliances, such as a smart speaker, a smart television (TV), or a smart refrigerator, and other devices, such as a smart vehicle, a smart kiosk, an Internet of things (IoT) device, a walking assist device (WAD), a drone, or a robot.

Accordingly, the total simulation time of a large-scale workload may be shortened, a large-scale workload may be split according to computational complexity of a localized model, and the utilization rate of homogeneous or heterogeneous hardware resources of a system for computational acceleration may be increased. Additionally, as a physical/chemical process evolves, a model-partition mapping for distributed computation may be dynamically updated.

Additionally, the electronic device 1000 may process the operations described with reference to FIG. 10.

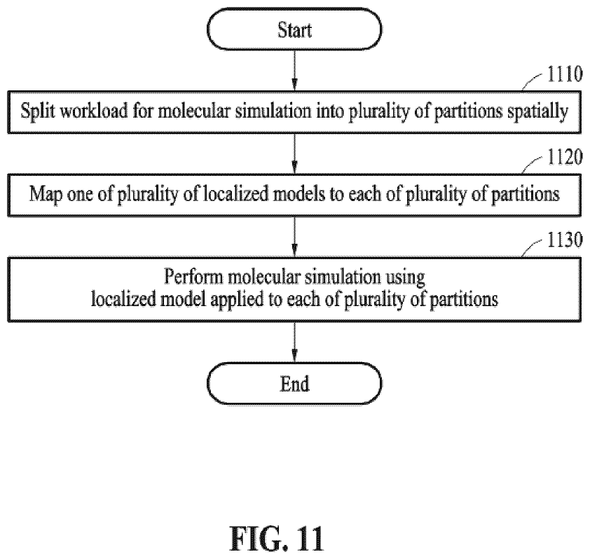

FIG. 11 illustrates a method of operating an electronic device, according to an embodiment.

In the following embodiments, operations may be performed sequentially but embodiments may not be necessarily limited thereto. For example, the order of the operations may change and at least two of the operations may be performed in parallel. Operations 1110 to 1130 may be performed by at least one component (e.g., at least one processor) of an electronic device.

In operation 1110, the electronic device may (e.g., spatially) split a workload for a molecular simulation into a plurality of partitions. In some cases, the plurality of partitions of the workload may correspond to a plurality of regions of a molecular structure.

In operation 1120, the electronic device may map one of a plurality of localized models to each of the plurality of partitions. In some cases, the electronic device may map a localized model to each of the plurality of partitions of the workload based on a corresponding region of the plurality of regions of the molecular structure.

In operation 1130, the electronic device may perform the molecular simulation based on the mapping (such as the mapping of operation 1120) of the localized model to each of the plurality of partitions of the workload.

The descriptions provided with reference to FIGS. 1 to 10 may apply to the operations shown in FIG. 11, and thus repeated descriptions may be omitted.

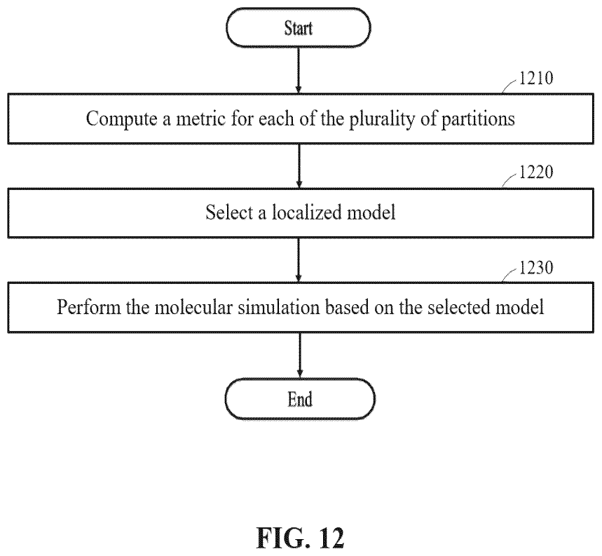

FIG. 12 illustrates a method of performing a molecular simulation, according to an embodiment.

In the following embodiments, operations may be performed sequentially but embodiments may not be necessarily limited thereto. For example, the order of the operations may change and at least two of the operations may be performed in parallel. Operations 1210 to 1230 may be performed by at least one component (e.g., at least one processor) of an electronic device.

In operation 1210, an electronic device may compute a metric for each of a plurality of partitions. In some cases, the metric may be computed of a workload for a molecular simulation process.

In operation 1220, the electronic device may select a localized model. In some cases, the localized model may be selected for each of the plurality of partitions based on the metric.

In operation 1230, the electronic device may perform the molecular simulation based on the selected localized model (such as the localized model selected in operation 1220) of each of the plurality of partitions of the workload.

The descriptions provided with reference to FIGS. 1 to 10 may also apply to the operations shown in FIG. 12, and thus repeated descriptions may be omitted.

The embodiments described herein may be implemented using a hardware component, a software component, and/or a combination thereof. A processing device may be implemented using one or more general-purpose or special-purpose computers, such as, for example, a processor, a controller and an arithmetic logic unit (ALU), a digital signal processor (DSP), a microcomputer, a field-programmable gate array (FPGA), a programmable logic unit (PLU), a microprocessor, or any other device capable of responding to and executing instructions in a defined manner. The processing device may run an operating system (OS) and one or more software applications that run on the OS. The processing device also may access, store, manipulate, process, and generate data in response to execution of the software. For purpose of simplicity, the description of a processing device is singular; however, one of ordinary skill in the art will appreciate that a processing device may include multiple processing elements and multiple types of processing elements. For example, the processing device may include a plurality of processors, or a single processor and a single controller. In addition, different processing configurations are possible, such as parallel processors.

The software may include a computer program, a piece of code, an instruction, or some combination thereof, to independently or collectively instruct or configure the processing device to operate as desired. Software and data may be stored in any type of machine, component, physical or virtual equipment, or computer storage medium or device capable of providing instructions or data to or being interpreted by the processing device. The software may also be distributed over network-coupled computer systems so that the software is stored and executed in a distributed fashion. The software and data may be stored in a non-transitory computer-readable recording medium.

The methods according to the above-described embodiments may be recorded in non-transitory computer-readable media including program instructions to implement various operations of the above-described embodiments. The media may also include, alone or in combination with the program instructions, data files, data structures, and the like. The program instructions recorded on the media may be those specifically designed and constructed for the purposes of embodiments, or they may be of the kind well known and available to those having skill in the computer software arts. Examples of non-transitory computer-readable media include magnetic media such as hard disks, floppy disks, and magnetic tape; optical media such as compact disc read-only memory (CD-ROM) discs and digital video discs (DVDs); magneto-optical media such as optical discs; and hardware devices that are specifically configured to store and perform program instructions, such as read-only memory (ROM), random access memory (RAM), flash memory, and the like. Examples of program instructions include both machine code, such as one produced by a compiler, and files containing higher-level code that may be executed by the computer using an interpreter.

The above-described hardware devices may be configured to act as one or more software modules in order to perform the operations of the above-described embodiments, or vice versa.

As described above, although the embodiments have been described with reference to the limited drawings, one of ordinary skill in the art may apply various technical modifications and variations based thereon. For example, suitable results may be achieved if the described techniques are performed in a different order, and/or if components in a described system, architecture, device, or circuit are combined in a different manner, and/or replaced or supplemented by other components or their equivalents.

Therefore, other implementations, other embodiments, and equivalents to the claims are also within the scope of the following claims.

The processes discussed above are intended to be illustrative and not limiting. One skilled in the art would appreciate that the steps of the processes discussed herein may be omitted, modified, combined, and/or rearranged, and any additional steps may be performed without departing from the scope of the invention. More generally, the above disclosure is meant to be exemplary and not limiting. Only the claims that follow are meant to set bounds as to what the present invention includes. Furthermore, it should be noted that the features and limitations described in any one embodiment may be applied to any other embodiment herein, and flowcharts or examples relating to one embodiment may be combined with any other embodiment in a suitable manner, done in different orders, or done in parallel. In addition, the systems and methods described herein may be performed in real time. It should also be noted, the systems and/or methods described above may be applied to, or used in accordance with, other systems and/or methods.

What is claimed is:

1. An electronic device comprising:
at least one processor; and
a memory configured to store instructions,
wherein at least one of the instructions, when executed by the at least one processor, causes the electronic device to:
split a workload for a molecular simulation into a plurality of partitions corresponding to a plurality of regions of a molecular structure, respectively;
map a localized model of a plurality of localized models to each of the plurality of partitions of the workload based on a corresponding region of the plurality of regions of the molecular structure; and
perform the molecular simulation based on the mapping of the localized model to each of the plurality of partitions of the workload.

2. The electronic device of claim 1, wherein:
the workload is split into the plurality of partitions based on a computational complexity of the plurality of localized models.

3. The electronic device of claim 1, wherein the instructions, when executed by the at least one processor, cause the electronic device to:
determine that a computational complexity of the localized model is greater than a computation threshold; and
split a corresponding partition of the plurality of partitions into a plurality of sub-partitions based on the determination.

4. The electronic device of claim 1, wherein the instructions, when executed by the at least one processor, cause the electronic device to:
compute a metric for a corresponding partition of the plurality of partitions;
determine whether a change in the metric is greater than a metric threshold; and
iteratively update the localized model based on the determination.

5. The electronic device of claim 4, wherein the instructions, when executed by the at least one processor, cause the electronic device to:
perform the molecular simulation for the corresponding partition using a tabular model based on an adjacent partition when the change in the metric is less than the metric threshold.

6. The electronic device of claim 4, wherein the metric comprises at least one of:
an accuracy of the localized model, an atom in the corresponding partition, a ghost atom in the corresponding partition, a radial distribution function (RDF), an angular distribution function (ADF), temperature and pressure of the corresponding partition, and a motion of atoms in the corresponding partition.

7. The electronic device of claim 1, wherein:
the plurality of localized models comprises localized models of different computational complexities.

8. The electronic device of claim 1, wherein:
the at least one processor comprises a plurality of homogeneous processors, and a higher computational complexity of a corresponding localized model results in a smaller size of a corresponding partition.

9. The electronic device of claim 1, wherein the instructions, when executed by the at least one processor, cause the electronic device to:
determine a processor among a plurality of heterogeneous processors; and
allocate the processor to the localized model based on a computational complexity of the localized model.

10. The electronic device of claim 1, wherein the instructions, when executed by the at least one processor, cause the electronic device to:
determine an absence of an atomic interaction in the corresponding partition; and
switch off an interaction parameter for the corresponding partition.

11. A method of operating an electronic device, the method comprising:
splitting a workload for a molecular simulation into a plurality of partitions corresponding to a plurality of regions of a molecular structure, respectively;
mapping a localized model of a plurality of localized models to each of the plurality of partitions of the workload based on a corresponding region of the plurality of regions of the molecular structure; and
performing the molecular simulation based on the mapping of the localized model to each of the plurality of partitions of the workload.

12. The method of claim 11, wherein
the workload is split into the plurality of partitions based on a computational complexity of the plurality of localized models.

13. The method of claim 11, further comprising:
determining that a computational complexity of the localized model is greater than a computation threshold;
splitting a corresponding partition of the plurality of partitions into a plurality of sub-partitions based on the determination.

14. The method of claim 11, further comprising:
computing a metric for a corresponding partition of the plurality of partitions;
determining whether a change in the metric is greater than a metric threshold; and
iteratively updating the localized model based on the determination.

15. The method of claim 14, further comprising:
performing the molecular simulation for the corresponding partition using a tabular model based on an adjacent partition when the change in the metric is less than the metric threshold.

16. The method of claim 14, wherein the metric comprises at least one of:
an accuracy of the corresponding localized model, an atom in the corresponding partition, a ghost atom in the corresponding partition, a radial distribution function (RDF), an angular distribution function (ADF), temperature and pressure of the corresponding partition, and a motion of atoms in the corresponding partition.

17. The method of claim 11, wherein
the plurality of localized models comprises localized models of different computational complexities.

18. A non-transitory computer-readable storage medium storing instructions that, when executed by a processor, cause the processor to perform the method of claim 11.

19. A method comprising:
computing a metric for each of a plurality of partitions of a workload for a molecular simulation;
selecting a localized model for each of the plurality of partitions based on the metric; and
performing a molecular simulation using the selected localized model for each of the plurality of partitions.

20. The method of claim 19, further comprising:
iteratively updating the metric at each of a plurality of stages of the molecular simulation; and
iteratively updating the localized model based on the updated metric.
